SEQUENCE LISTING (7 sequences)

SEQ ID NO: 1 - DNA, 275 bp, Bacillus subtilis
  misc_feature (1)..(275): Bacillus subtilis strain NEB4 16S ribosomal RNA gene partial sequence (AF406704)
gacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccag

SEQ ID NO: 2 - DNA, 275 bp, Bacillus subtilis; identical to SEQ ID NO: 1
  misc_feature (1)..(275): Bacillus subtilis strain NEB5 16S ribosomal RNA gene partial sequence (AF406705)

SEQ ID NO: 3 - DNA, 277 bp, Bacillus thuringiensis
  misc_feature (1)..(277): Bacillus thuringiensis strain NEB17 16S ribosomal RNA gene partial sequence (AF406706)
aatggattaagagcttgctcttatgaagttagcggcggacgggtgagtaacacgtgggtaacctgcccataagactgggataactccgggaaaccggggctaataccggataacattttgaactgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccag

SEQ ID NO: 4 - DNA, 20 bp, Artificial
  Primer 27f
agagtttgatcmtggctcag

SEQ ID NO: 5 - DNA, 22 bp, Artificial
  Primer 1492r
tacggytaccttgttacgactt

SEQ ID NO: 6 - DNA, 41 bp, Artificial
  Primer BhvF1
tgtaaaacgacggccagtgcctaatacatgcaagtcgagcg

SEQ ID NO: 7 - DNA, 39 bp, Artificial
  Primer BhvR1
caggaaacagctatgaccactgctgcctcccgtaggagt

(19) United States
(12) Patent Application Publication
(10) Pub. No.: US 2003/0228679 A1
(43) Pub. Date: Dec. 11, 2003

(54) Compositions and methods for increasing plant growth by inoculation with bacillus strains

(76) Inventors: Donald L. Smith, Ste.-Anne-de-Bellevue (CA); Brian Driscoll, Pierrefonds (CA); Yuming Bai, Cambridge (CA)

Correspondence Address: SMART & BIGGAR, P.O. BOX 2999, STATION D, 55 METCALFE STREET, SUITE 900, OTTAWA, ON, K1P5Y6, CA

(21) Appl. No.: 10/396,446

(22) Filed: Mar. 26, 2003

Related U.S. Application Data
(60) Provisional application No. 60/367,480, filed on Mar. 27, 2002.

(51) Int. Cl. C12N 7/00; C12N 7/01

(52) U.S. Cl. 435/235.1

(57) ABSTRACT

The invention provides inoculants for increasing plant growth, comprising plant growth promoting bacteria selected from the group consisting of plant growth promoting bacteria of the species Bacillus subtilis and plant growth promoting bacteria of the species Bacillus thuringiensis, or a combination thereof, and methods for using the inoculants for increasing plant growth. Preferably the plant growth promoting bacteria are selected from the group consisting of B. subtilis having the identifying characteristics of B. subtilis strain NEB4, B. subtilis having the identifying characteristics of B. subtilis strain NEB5, and B. thuringiensis having the identifying characteristics of B. thuringiensis strain NEB17.

### CROSS-REFERENCE TO RELATED APPLICATIONS

This application claims the benefit of United States provisional patent application No. 60/367,480, filed Mar. 27, 2002, which is incorporated by reference herein.

### FIELD OF THE INVENTION

The invention relates to microbial inoculants for improving plant growth.

### BACKGROUND OF THE INVENTION

Legume-rhizobia symbioses actively fix nitrogen and are critical to agricultural crop production. Enhancement of legume nitrogen fixation by co-inoculation of rhizobia with some plant growth promoting bacteria (PGPB) is a practical way to improve nitrogen availability in sustainable agriculture production systems. Accordingly, there is great interest in identifying new PGPB and methods for improving plant growth with PGPB. Most of the PGPB strains tested by co-inoculation with Rhizobium or Bradyrhizobium species are general rhizobacteria. However, in the recent years, other bacteria, such as endophytic bacteria, have drawn attention as a group of potential PGPB.

### BRIEF SUMMARY OF THE INVENTION

In one aspect, the invention provides a method for increasing plant growth, comprising inoculating a plant with plant growth promoting bacteria selected from the group consisting of plant growth promoting bacteria of the species Bacillus subtilis and plant growth promoting bacteria of the species Bacillus thuringiensis, or a combination thereof.

As used herein, the term “increasing plant growth” includes, without limitation, increasing plant weight, increasing nodule number, increasing nodule weight, increasing nitrogen fixation, increasing total biomass, and increasing grain yield.

In a preferred embodiment, the bacteria of the genus Bacillus are selected from the group consisting of B. subtilis having the identifying characteristics of B. subtilis strain NEB4, B. subtilis having the identifying characteristics of B. subtilis strain NEB5, and B. thuringiensis having the identifying characteristics of B. thuringiensis strain NEB17.

In one embodiment, the plants are also inoculated with nitrogen-fixing rhizobacteria. In a preferred embodiment, the nitrogen-fixing rhizobacteria comprise bacteria of the genus Bradyrhizobium, preferably the species Bradyrhizobium japonicum.

In one embodiment, the plant is a nitrogen-fixing plant such as a legume, e.g. a soybean.

The invention also provides an inoculant for increasing plant growth, comprising plant growth promoting bacteria selected from the group consisting of Bacillus subtilis and Bacillus thuringiensis, or a combination thereof. In a preferred embodiment, the bacteria of the genus Bacillus are selected from the group consisting of B. subtilis having the identifying characteristics of B. subtilis strain NEB4, B. subtilis having the identifying characteristics of B. subtilis strain NEB5, and B. thuringiensis having the identifying characteristics of B. thuringiensis strain NEB17.

In one embodiment, the inoculant further comprises nitrogen-fixing rhizobacteria, e.g. nitrogen-fixing rhizobacteria of the genus Bradyrhizobium, such as bacteria of the species Bradyrhizobium japonicum.

In another aspect, the invention provides a kit for increasing plant growth, comprising an inoculant as described above and instructions for using the inoculant for increasing plant growth.

In another aspect, the invention provides a biologically pure culture of plant growth promoting bacteria selected from the group consisting of:

(a) a biologically pure culture of Bacillus subtilis having the identifying characteristics of B. subtilis strain NEB4;

(b) a biologically pure culture of Bacillus subtilis, having the identifying characteristics of B. subtilis strain NEB5; and

(c) a biologically pure culture of Bacillus thuringiensis having the identifying characteristics of B. thuringiensis strain NEB17.

As used herein, the term “biologically pure culture” means a culture descended from a single cell.

### BRIEF DESCRIPTION OF THE SEVERAL VIEWS OF THE DRAWINGS

FIG. 1 illustrates the effects of NEB strains on soybean plants co-inoculated with B. japonicum. Plants were cultured in growth pouches with N-free Hoagland's solution, harvested 55 days after inoculation, and then nodule number (A), nodule weight (B), and plant weight (C) were determined. Control plants (532C) were inoculated with B. japonicum 532C alone, all other plants were inoculated with B. japonicum 532C plus one of the NEB strains, as indicated. The bars represent the mean values (n=6), and the letters above each bar indicate significance at the P=0.05 level.

FIG. 2 illustrates growth of NEB17 (A, D), NEB5 (B, E), and NEB4 (C, F) in Ashbey's broth with different carbon and nitrogen sources. Media contained either mannitol (A, B, C), or glucose (D, E, F) as carbon source. The Ashbey's broth was nitrogen-free (closed circles), or supplemented with either 0.5 g/l NH₄NO₃ (open circles), or 1 g/l peptone (closed triangles). Note that peptone is a complex nutrient containing both carbon and nitrogen compounds. Data are the mean (n=3) optical density, and error bars are the standard deviation.

FIG. 3 illustrates phylogenetic relationships between NEB4, NEB5, NEB17, and representative Bacillus species based on 16S rDNA HV sequences. The dendrogram was generated by the neighbor-joining method, with Kimura distances, and is rooted to the out-group A. acidoterrestris. Nodes with greater than 70% bootstrap support (1000 replications) are indicated. The bar represents 0.02 nucleotide substitutions per site. Accession numbers are reported in Example 1.

FIG. 4 illustrates the effects of co-inoculation of Bacillus strains on nodule number and nodule weight (I), root weight and shoot weight (II) of greenhouse grown soybean plants in the pot experiment. Inoculant: 532C, Bradyrhizobium japonicum 532C; KMB, 532C+King's Medium B; NEB4, NEB5 and NEB17, co-inoculation of 532C and Bacillus subtilis NEB4, B. subtilis NEB5 and B. thuringiensis NEB17, respectively. Bars associated with the same letter are not different (P=0.05) by an ANOVA protected LSD test. The small letters are for the comparisons of nodule number (I) and root weight (II) among the inoculants. The capital letters are for the comparisons of nodule weight (I) and shoot weight (II) among the inoculants. n=5.

FIG. 5 illustrates monthly average temperature (I) and precipitation (II) in growing season.

FIG. 6 illustrates seed and total nitrogen yield for field grown (Year 1 and Year 2) soybean plants co-inoculated with three Bacillus strains and the control. Inoculant: Control, no NEB co-inoculated; NEB4, NEB5 and NEB17, co-inoculation of Bacillus subtilis NEB4, B. subtilis NEB5 and B. thuringiensis NEB17, respectively. Bars associated with the same letter are not different at P=0.05 level for total N and seed N in Year 1 and for total N in Year 2, and at P=0.07 for seed N in Year 2, by an ANOVA protected LSD test. n=9.

### DETAILED DESCRIPTION OF THE INVENTION

The plant growth promoting bacteria is selected from the group consisting of B. subtilis and B. thuringiensis or a combination thereof. Preferred are B. subtilis or B. thuringiensis strains having a 16S ribosomal RNA hypervariant region possessing at least 60%, preferably 70%, 80%, 85%, 90%, 95%, 98%, or 99% nucleotide identity to the partial 16S rRNA sequence depicted in SEQ ID NO: 1, 2 or 3 as calculated using the BLASTn algorithm (version BLASTN 2.2.5; Nov-16-2002) available through the National Center for Biotechnology Information (www-ncbi.nlm.nih.gov/) at default settings, as described in Altschul et al. (1997). Particularly preferred are B. subtilis having the identifying characteristics of B. subtilis strain NEB4 or NEB5, and B. thuringiensis having the identifying characteristics of B. thuringiensis strain NEB17. Particularly preferred “identifying characteristics” include 16S rRNA gene sequence. For example, as discussed herein, B. subtilis strain NEB4 has a partial 16S rRNA gene sequence as set forth in SEQ ID NO: 1. Thus, e.g., a strain of Bacillus subtilis having plant growth promoting activity and a partial 16S gene sequence as set forth in SEQ ID NO: 1 is understood to have the “identifying characteristics” of B. subtilis strain NEB4.

The selection of the nitrogen-fixing rhizobacteria is not critical to the invention, and any nitrogen fixing rhizobacteria may be used. As used herein, the term “nitrogen fixing rhizobacteria” means bacteria of the family Rhizobiaciae that are able to enter into a symbiotic nitrogen fixing relationship with a leguminous plant, and supply the plant with nitrogen. Most nitrogen fixing rhizobacteria are members of the genera Bradyrhizobium, Rhizobium, Sinorhizobium, and Azorhizobium. Many suitable nitrogen fixing rhizobacteria are known to the those of skill in the art, and are available commercially. Particularly preferred nitrogen fixing rhizobacteria include rhizobacteria of the genus Bradyrhizobium, particularly B. japonicum.

The methods and compositions of the invention are useful for increasing growth in a wide range of plants, including, without limitation, legumes, non-legumes, cereals, oilseeds, fiber crops, starch crops and vegetables. Non-limiting examples of legumes include soybeans; peanuts; chickpeas; all the pulses, including peas and lentils; all the beans; major forage crops, such as alfalfa and clover; and many more plants of lesser agricultural importance, such as lupines, sainfoin, trefoil, and even some small tree species. Non-limiting examples of cereals include corn, wheat, barley, oats, rye and triticale. Non-limiting examples of oilseeds include canola and flax. Non-limiting examples of fiber crops include hemp and cotton. Non-limiting examples of starch crops include potato, sugar cane and sugar beets. Non-limiting examples of vegetables include carrots, radishes, cauliflower, broccoli, peppers, lettuce, cabbage, peppers, celery and Brussels sprouts.

Techniques for applying inoculants to plants are known in the art, including appropriate modes of administration, frequency of administration, dosages, et cetera. Typically, inoculants are in a liquid or powdered form. Suitable auxiliaries, such as carriers, diluents, excipients, and adjuvants are known in the art. For example, dry or semi-dry powdered inoculants often comprise the microorganism(s) of interested dispersed on powdered peat, clay, other plant material, or a protein such as casein. The inoculant may include or be applied in concert with other standard agricultural auxiliaries such as fertilizers, pesticides, or other beneficial microorganisms.

The inoculant may be applied to the soil prior to, contemporaneously with, or after sowing seeds, after planting, or after plants have emerged from the ground. The inoculant may also be applied to seeds themselves prior to or at the time of planting (e.g. packaged seed may be sold with the inoculant already applied). The inoculant may also be applied to the plant after it has emerged from the ground, or to the leaves, stems, roots, or other parts of the plant.

The methods and compositions of the invention may be used in so-called “virgin soils” which do not contain an indigenous population of PGPB such as nitrogen fixing rhizobia. This may occur e.g. where nitrogen-fixing legume crops have not previously or recently been grown. In such instances, the inclusion in the inoculant of nitrogen-fixing rhizobia is particularly beneficial. In instances in which the soil already contains a substantial population of nitrogen-fixing rhizobia, an inoculant containing only plant growth promoting bacteria of the genus Bacillus may be preferred.

Inoculants may contain only one plant growth promoting bacterial strain of the genus Bacillus or may contain combinations of different Bacillus strains. One or more strains of nitrogen-fixing rhizobacteria or other beneficial microorganisms may also be present.

Kits containing inoculants of the invention will typically include one or more containers of the inoculant, and printed instructions for using the inoculant for promoting plant growth. The kit may also include tools or instruments for reconstituting, measuring, mixing, or applying the inoculant, and will vary in accordance with the particular formulation and intended use of the inoculant.

Further details concerning the preparation of bacterial inoculants and methods for inoculating plants with bacterial inoculants are found in e.g. U.S. Pat. Nos. 5,586,411; 5,697,186; 5,484,464; 5,906,929; 5,288,296; 4,875,921; 4,828,600; 5,951,978; 5,183,759; 5,041,383; 6,077,505; 5,916,029; 5,360,606; 5,292,507; 5,229,114; 4,421,544; and 4,367,609, each of which is incorporated herein by reference.

Unless defined otherwise, all technical and scientific terms used herein are intended to have the same meaning as is commonly understood by one of ordinary skill in the relevant art.

As used herein, the singular forms “a,” “an”, and “the” include the plural reference unless the context clearly dictates otherwise.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### EXAMPLE 1

This Example illustrates the isolation of plant-growth promoting Bacillus strains from soybean root nodules.

Materials and Methods

Bacterial Strains and Growth Conditions

Bradyrhizobium japonicum 532C is a Hup-strain adapted to Canadian soils (Hume and Shelp 1990). Wild-type strains of Staphylococcus aureus and Bacillus cereus were from the Microbiology Unit collection, Department of Natural Resource Sciences, McGill University. Bradyrhizobium japonicum was cultured at 28° C. using yeast extract mannitol (YEM; Vincent 1970). NEB strains were cultured at 28° C. using King's Medium B (Atlas 1995), or at 30° C. using Luria Bertani (LB) broth (Sambrook et al. 1989), or Ashbey's nitrogen-free medium (Atlas 1995) with different combinations of the following; mannitol (15 g/l), dextrose (15 g/l), NH₄NO₃ (0.5 g/l), proteose peptone (1 g/l; Anachemica Canada, Inc., Montreal QC), and yeast extract (1 g/l; Anachemica). Liquid cultures were grown in flasks or test tubes, with rotary agitation (200 rpm), and plates were prepared by solidifying the media with 1.5% [w/v] agar (Anachemica). Culture densities were estimated by optical density by A₆₂₀ for B. japonicum, or A₄₂₀ for the NEB strains (Dashti et al. 1997).

Isolation of Endophytic Bacteria From Surface-Sterilized Nodules

Twenty vigorous soybean [Glycine max. L. Merr] seedlings at the R3 stage (Fehr et al. 1971) were selected from five fields at the A. E. Lods Agronomy Research Center, Macdonald Campus, McGill University. The fields had been sown with soybean cultivars OAC Bayfield and OAC Maple Glen, and inoculated with B. japonicum 532C, as described (Dashti et al. 1997). The roots were washed thoroughly with tap water, and 80 healthy nodules were detached along with a portion of the root. The nodules were placed into sterilized flasks and were surface-sterilized by rinsing with 95% ethanol for 15 sec, and then with acidified 0.1% HgCl₂ solution for 3-5 min, depending on the size of the nodule. The nodules were then rinsed with three cycles of 4-5 changes of sterile H₂O, followed by soaking in sterile H₂O for 15 min.

Twenty nodules, four from each of the five fields, were placed into separate sterile Eppendorf tubes with 1 ml of sterile H₂O. To confirm nodule surface sterility, the tubes were vortexed (2 min), 0.1 ml of the surface-wash water was spread on YEM plates, and the plates were incubated at 28° C. for 4 days. Immediately following surface-sterilization, the nodules were crushed aseptically, nodule contents were streaked onto YEM plates, and the plates were incubated at 28° C. Non-Bradyrhizobium colonies were chosen on the basis of colony morphology and growth rate. After four days, non-Bradyrhizobium colonies were picked, and then were purified by single-colony streaking on three successive King's Medium B plates. A total of 14 strains with distinct colony morphologies (three strains from one nodule, two each from three nodules, and one each from five nodules) were kept for further study. Isolates were only retained from nodules that were confirmed to have been surface-sterilized. The putative nodule endophyte strains were designated as NEB (non-Bradyrhizobium endophytic bacteria).

Soybean Cultivation in Growth Pouches

Growth pouch experiments were arranged following a completely randomized split plot design with three replicates per inoculation treatment (Mead et al. 1993). Soybean (cultivar OAC Bayfield) seeds were surface-sterilized (2% NaOCl, 4 ml/l Tween 20, 3 minutes), rinsed with several changes of sterile H₂O, and then germinated in trays of vermiculite in a greenhouse. The greenhouse conditions were: air temperature 25±1° C., with supplemental illumination of 300 μmol/m²/s via high pressure sodium lights (P. L. Light System Canada) for a photoperiod of 16:8 h (day:night). Single four-day-old healthy seedlings at the VE stage (Fehr et al. 1971) were transplanted into each growth pouch (15×16 cm, Mega International, Minneapolis, Minn.) and suspended in a 25° C. water bath in the greenhouse. The plants were watered as needed with N-free Hoagland's solution, in which Ca(NO₃)₂ and KNO₃ were replaced with CaCl₂, K₂HPO₄ and KH₂PO₄, as recommended (Hoagland and Arnon 1950). Six days following transplantation, the seedlings were inoculated with 108 cells from late-log phase cultures of the NEB strains (King's Medium B), 72h sub-cultures of B. japonicum (YEM), or co-inoculated with combinations of both. Control plants were inoculated with 1 ml of sterile distilled H₂O or suitably-diluted sterile King's Medium B. Inoculation with the medium had no effect on plant growth or nodulation relative to inoculation with sterile distilled H₂O.

Plants were harvested 55 days following inoculation, and nodule number, nodule dry weight, root dry weight, and shoot dry weight data were collected, each on a per plant basis. Dry weight data were determined from samples dried at 70° C. for a minimum of 48 h. Plant dry weight values were the sum of shoot plus root dry weight values for each plant. When analysis of variance indicated differences among means, comparisons among the treatment means were conducted with an ANOVA protected least significance difference (LSD) test (Steel and Torrie 1980).

The three strains (NEB4, NEB5, NEB17) that had positive effects on soybean growth and/or nodulation when co-inoculated with B. japonicum 532C, were tested with soybean plants, as above, in the absence of B. japonicum. Control plants were inoculated with 1 ml of sterile distilled H₂O. Nodule number and plant dry weight were determined as above, and the nitrogen content of dried plants (shoot plus root) was determined using the Kjeldahl method (Kjeltec system, with Digestion System 20, and a 1002 Distilling Unit, Tecator AB, Hoganas, Sweden), as previously described (Bremner 1965). Control values for plant dry weight and nitrogen content were 864±68 mg/plant and 11.1 ±1.4 mg/plant, respectively (mean±SD, n=6).

Phenotypic Characterization of NEB Strains

NEB4, NEB5 and NEB17 cells were harvested from 24 h King's Medium B plates for cytological staining and microscopy. The cultures were tested for the presence of spores using the Schaeffer-Fulton staining method, and for Gram reaction. As all three strains were found to be Gram positive, they were assayed for carbon utilization using Biolog GP Microplates (Biolog Inc., Hayward, Calif.), following the manufacturer's instructions. Staphylococcus aureus and Bacillus cereus were used as controls. All strains were cultured on plates of Biolog Universal Growth Medium (BUGM; Biolog Inc.) plus 1% [w/v] glucose, at 30° C. for 9 h. Glucose was added to the BUGM in an attempt to limit the degree of sporulation, as directed by the manufacturer for dealing with putative Bacillus species. Aliquots (150 μl) of the cell suspensions were distributed into each of the 96 wells, and then the Microplates were incubated at 30° C. Colourimetric changes were measured by determining the A₅₉₅, after 4 h and 24 h, using a 3550-UV Microplate Reader (BioRad Laboratories, Mississauga, ON). Readings were standardized against the control well containing no carbon source. Standardized absorbance values greater than 0.1 were scored as positive. Putative identifications were made using MicroLog1 v. 3.50 software plus database (Biolog), and only similarity index (SIM) values above 0.5 were considered significant for identification purposes (Biolog).

Extraction of Plasmid and Bacterial Genomic DNA

Genomic DNA was extracted from cultures of NEB4, NEB5, and NEB17 grown to stationary phase in LB broth at 30° C., using the standard lysozyme/SDS/Pronase protocol (Sambrook et al. 1989). The DNA was purified using DNeasy Tissue kits (Qiagen, Mississauga, ON). Plasmid DNA was isolated from cultures grown in LB plus ampicillin (50 μg/ml) at 37° C., using QIAprep spin miniprep kits (Qiagen) according to the manufacturer's instructions. Agarose gel electrophoresis (0.8% agarose, TAE buffer pH 8.0) and staining with 0.5 mg/l ethidium bromide was done as previously described (Sambrook et al. 1989). DNA concentrations were estimated relative to the HindIII-digested lambda DNA standard (Gibco-BRL, Life Technologies, Burlington, ON) using an AlphaImager (Alpha Innotech, Mississauga, ON). PCR products, and plasmid DNA to be used as template in DNA sequencing reactions, were excised from agarose gels and purified using QIAEX II gel extraction kits (Qiagen).

PCR Amplification and DNA Sequencing

The complete 1.6 kb 16S rDNA region was amplified using the universal bacterial 16S rDNA primers 27f [5′-AGA GTT TGA TCM TGG CTC AG] (SEQ ID NO: 4), and 1492r [5′-TAC GGY TAC CTT GTT ACG ACT T] (SEQ ID NO: 5)(Ritchie et al. 1997). Primers BhvF1 [5′-TGT AAA ACG ACG GCC AGT GCC TAA TAC ATG CAA GTC GAG CG] (SEQ ID NO: 6), and BhvR1 [5′-CAG GAA ACA GCT ATG ACC ACT GCT GCC TCC CGT AGG AGT] (SEQ ID NO: 7), were used to amplify approximately 350 bp containing the hypervariant (HV) region of Bacillus 16S rDNA (Goto et al. 2000). PCR reactions (50 μl) contained: 25 to 50 ng of purified genomic DNA; 10 pmol of each primer; PCR buffer (Gibco-BRL); 1.5 mM Mg²⁺ (Gibco-BRL); and 200 μM dNTPs (Roche, Laval, QC, Canada). Template DNA was denatured at 94° C. for 3 min, then 2.5 U Taq DNA polymerase (Gibco-BRL) was added, and the reaction was cycled 30 times as follows: denaturation for 1 min at 92° C.; annealing for 1 min 60° C.; extension for 1 min at 72° C. This was followed by a final extension for 5 min at 72° C. A PTC-100 thermocycler (MJ Research, Waltham, Mass.) was used.

PCR products were ligated into the vector pGEM-T Easy, and ligation products were transformed into CaCl₂-competent E. coli DH5α cells, using the materials and protocols supplied with the vector (Promega Inc., Madison, Wis., USA). Plasmid DNA was isolated from positive clones, and purified prior to sequencing, as described above. DNA sequencing was done using the ABI PRISM Big Dye Terminator Cycle Sequencing Ready Reaction Kit (PE Applied Biosystems, Mississauga, ON), and standard T7 and SP6 promoter sequencing primers (Gibco-BRL). Sequencing reactions were run on an ABI PRISM 310 Genetic Analyzer (PE Applied Biosystems). Nucleotide sequences were compiled using Sequencher v. 3.0 (Gene Codes Corporation, Inc., Ann Arbor, Mich.). The NEB4 (275 nucleotides), NEB5 (275 nucleotides), and NEB17 (277 nucleotides) 16S rRNA gene HV sequences were deposited in GenBank under accession numbers AF406704 (SEQ ID NO: 1), AF406705 (SEQ ID NO: 2), and AF406706 (SEQ ID NO: 3), respectively.

Phylogenetic Analysis

DNA sequences were compared to the nr nucleotide databases using the standard nucleotide-nucleotide BLAST (blastn) search algorithm (Altschul et al. 1997). Phylogenetic analysis was done using MacVector v. 7.0 (Oxford Molecular Ltd., Genetics Computer Group, Madison Wis.). Nucleotide sequences were aligned using the CLUSTAL W algorithm (Thompson et al. 1994). Phylogenetic trees were reconstructed by the neighbor-joining method (Saitou and Nei 1987), using the distance matrix from the alignment. Distances were calculated using both the Kimura (Kimura 1980) and Tamura-Nei (Tamura and Nei 1993) methods. Gaps were ignored, no gamma correction shape was specified, and for the Kimura method, the transition:transversion ratio was estimated by the algorithm (average=1.81). Phylogenetic trees were subjected to bootstrap analysis with 1000 replications (Felsenstein 1985). 16S rDNA sequence of the following strains (type strains, unless otherwise indicated) were obtained from GenBank (accession numbers in brackets): B. thermoglucosidasius (ABO21197); B. stearothermophilus (AB021196); B. weihenstephanensis (AB021199); B. mycoides (AB021192); B. thuringiensis WS2625 (Z84587); B. mojavensis (AB021191); B. vallismortis (AB021198); B. atrophaeus (AB021181); B. subtilis (X60646); B. carboniphilus (AB021182); B. psychrosaccharolyticus (AB021195); B. marinus (AB021190); B. flexus (AB021185); B. niacini (AB021194); B. megaterium (D16273); and the out-group, the Gram positive bacterium Alicyclobacillus acidoterrestris DSM 3922T (X60742).

Results

Isolation of Bacterial Strains From Soybean Root Nodules

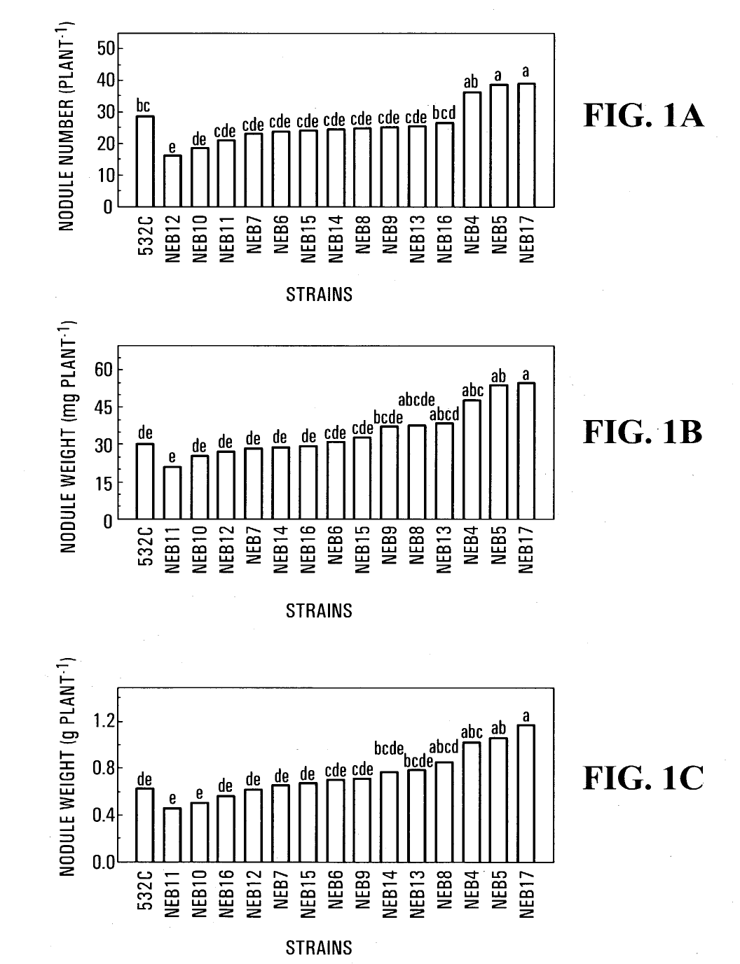

We wished to isolate non1-Bradyrhizobium bacteria from within soybean root nodules. To reduce the possibility of isolating rhizobacteria from the surface of the nodules, only nodules that were confirmed to have been surface-sterilized were used. As Bradyrhizobia require nearly a week to form colonies on YEM plates, colonies that arose from crushed nodule contents were picked after an incubation of only four days, and no colonies that had a similar morphology to the soybean endosymbiont, B. japonicum, were chosen. Colonies of putative non-Bradyrhizobium endophytes (NEB) were observed on plates from nine out of 17 crushed nodules. Of the 17 NEB strains isolated, 14 had distinct colony morphologies, and so were selected for further study. Effects of the NEB strains on the growth of soybean plants Soybean seedlings were co-inoculated with B. japonicum 532C and each of the 14 distinct NEB isolates. Plant weight, nodule number and nodule weight were determined 55 days after inoculation (FIG. 1.). While the majority of the isolates had no significant effects on soybean growth and development, three (NEB4, NEB5 and NEB17) appeared to have positive effects. Plants co-inoculated with these strains had significantly higher nodule and plant weights, and NEB5 and NEB17 seemed to increase nodule number per plant. These strains also had positive effects on soybean growth when the root zone temperature was lowered (results not shown). Isolates NEB10, NEB11 and NEB12, seemed to be the poorest performers overall, with some significant decreases in plant weight and nodule number compared to the control. The remaining isolates had no significant effects on soybean growth or modulation. All further experiments were limited to the soybean-growth promoting strains NEB4, NEB5, and NEB17. Once it had been determined that the eleven other isolates had no positive effects on soybeans, they were discarded.

There was no evidence that the positive soybean-growth effects of NEB4, NEB5, and NEB17 were as a result of supplying the plants with fixed nitrogen. The strains were each inoculated onto soybean seedlings, as above, but in the absence of B. japonicum 532C. None of these strains were able to form root nodules with soybean, and the plants appeared chlorotic and stunted, similar to uninoculated control plants. Neither the plant weights nor their nitrogen contents were significantly different from uninoculated control plants (results not shown).

Phenotypic Characterization the NEB Strains

Distinct colony morphologies were observed for NEB4, NEB5 and NEB17 on King's Medium B plates. NEB4 and NEB5 colonies both had slimy capsules, and produced red, water-soluble, pigments. NEB17 colonies had a waxy appearance, with no pigment. All three strains were determined to be Gram positive spore-forming rods.

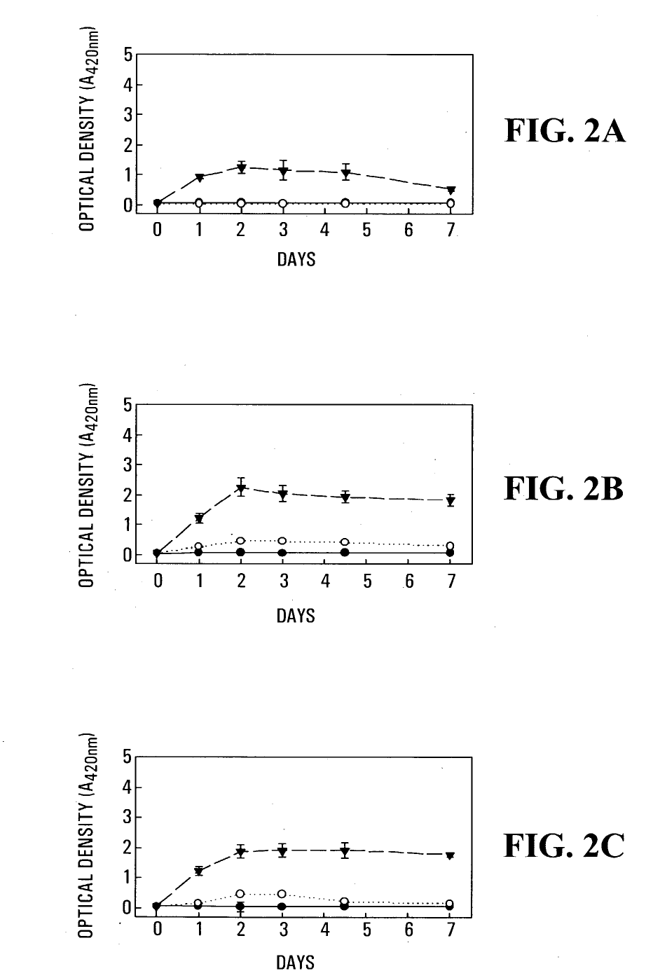

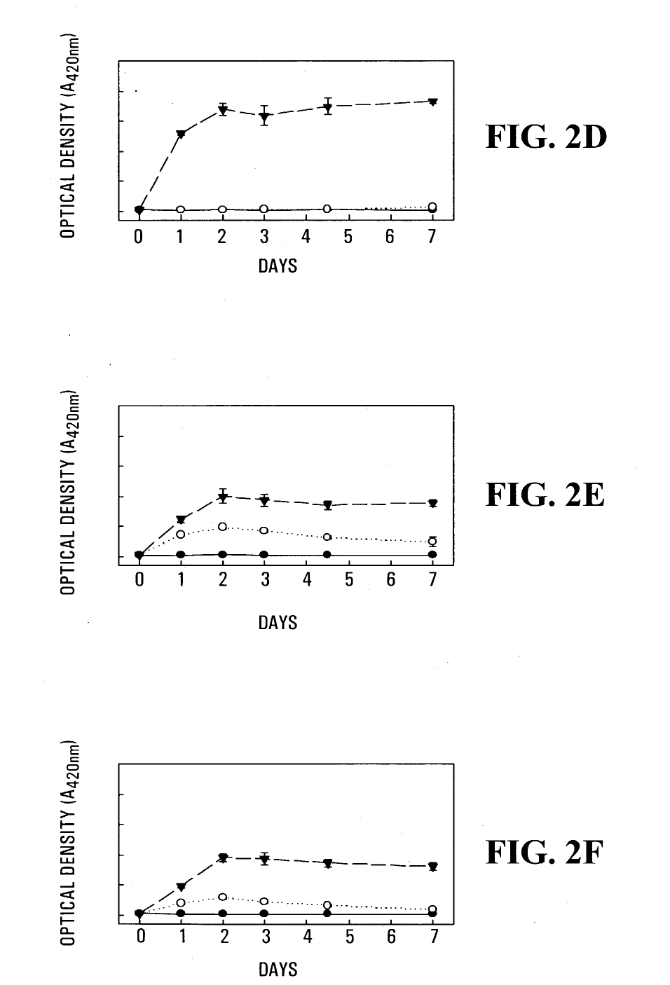

NEB4, NEB5, and NEB17 cultures showed no significant growth after 7 days in Ashbey's nitrogen-free broth (FIG. 2), or after 30 days on plates of the same medium (results not shown). We therefore concluded that these strains were unable to fix nitrogen aerobically. All three strains responded best when nitrogen was provided in complex form, with identical growth with either peptone (FIG. 2) or yeast extract (results not shown). With NH₄NO₃ as sole nitrogen source in Ashbey's broth, with either carbon source, NEB4 and NEB5 grew poorly, and NEB17 grew very poorly. With respect to carbon sources, NEB4 and NEB5 showed similar growth when supplied with either mannitol or dextrose, whereas NEB17 showed much better growth with dextrose. The results for growth of these strains on Ashbey's plates with the same additions mirrored those for liquid cultures (results not shown).

The NEB strains could not be identified at the species level using the Biolog system, due to a very high percentage of false-positive results. This result was anticipated, however, as spore-forming bacteria, such as Bacillus species, frequently yield false-positives in Biolog tests. This phenomenon is discussed in the Biolog technical literature, and has been observed by others (Baillie et al. 1995). Despite numerous attempts, the SIM values for the NEB strains, and the B. cereus control (0.315), were below the threshold of 0.5 acceptable for species identification. The SIM value for the (non spore-forming) S. aureus control was, however, 0.563. The Biolog database matches with the highest SIM values were to B. subtilis for both NEB4 (0.242) and NEB5 (0.426). For NEB17, the best matches were to B. mycoides (0.483), B. cereus (0.417), and B. thuringiensis (0.417). Therefore, while these tests indicated that the NEB strains were Bacillus species, they did not provide identifications at the species level.

Analysis of 16S rDNA Sequences

Single PCR products of the expected size (1.6 kb) were amplified from NEB4, NEB5 and NEB17 using bacterial 16S rDNA primers. The PCR products were cloned, and single strand sequences of 400-450 nucleotides from both ends of each clone were determined. The NEB4 and NEB5 sequences were identical to each other. BLAST comparisons, done to verify that the clones contained 16S rDNA, revealed that the NEB17 sequences had very high homology to the 5′ and 3′ ends of the B. thuringiensis WS2625 16S rRNA gene, and that the NEB4 and NEB5 sequences had very high homology to the 5′ and 3′ ends of B. subtilis 16S rRNA genes.

As all indications were that the three NEB strains were Bacillus species, we utilized PCR primers designed to amplify the hypervariant (HV) region of Bacillus 16S rDNA (Goto et al. 2000). The PCR amplifications yielded single PCR products of the expected size, approximately 350 bp, for each strain. The PCR products were cloned, and nucleotide sequences were generated for both strands. The NEB4 and NEB5 HV sequences (275 nucleotides) were identical, and were identical to those of thirteen B. subtilis strains. The NEB17 HV sequence (277 nucleotides) was identical to B. thuringiensis strain WS2625.

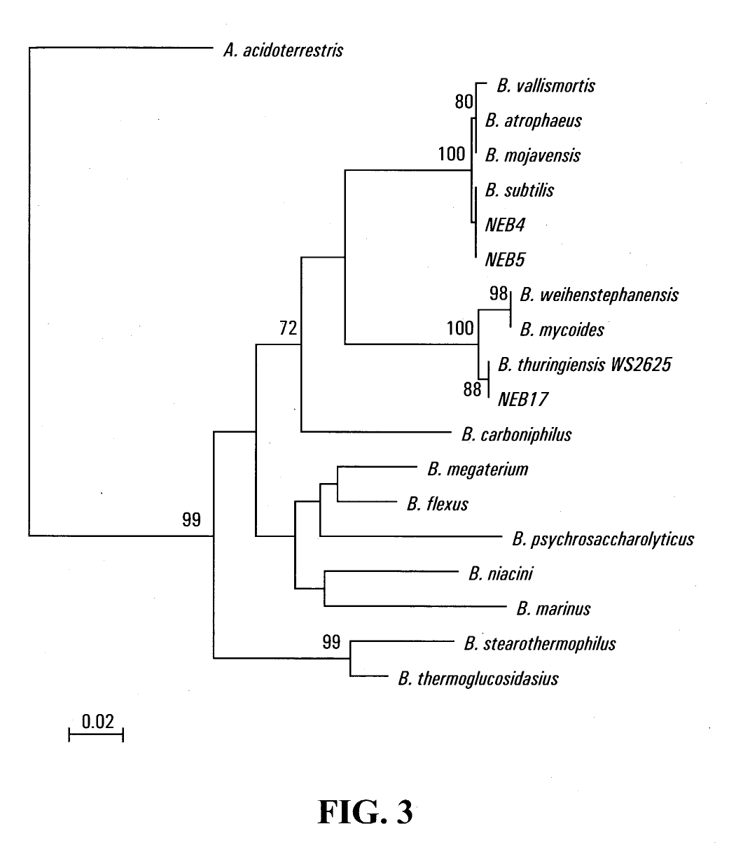

A neighbor-joining dendrogram was generated using the HV sequences from the NEB strains and representative Bacillus sequences from GenBank (FIG. 3). As expected, NEB4 and NEB5 clustered with B. subtilis, and NEB17 clustered with B. thuringiensis WS2625. The separation of the NEB4/NEB5/B. subtilis cluster from the B. vallismortis/B. mojavensis/B. atrophaeus cluster was supported by a bootstrap value of 100%. The separation of the NEB17/B. thuringiensis WS2625 cluster from the B. weihenstephanensis/B. mycoides cluster also had 100% bootstrap support. The same tree topology and high bootstrap values were achieved using Tamura-Nei distances (results not shown). The phylogenetic relationships between species related to the NEB strains, and those between HV sequences of other Bacillus species, particularly those from the B. megaterium and B. stearothermophilus clusters, were reconstructed as previously reported (Goto et al. 2000).

### EXAMPLE 2

This Example illustrates enhanced soybean plant growth due to co-inoculation of Bacillus strains with Bradyrhizobium japonicum.

Materials and Methods

Preparation of the Bacterial Inoculants

This work was conducted with the soybean [Glycine max. (L.) Merr.] cultivar OAC Bayfield, coinoculated with Bradyrhizobium japonicum strains 532C or USDA110, and with one of the three endophytic bacterial strains: Bacillus subtilis NEB4 (NEB4), B. subtilis NEB5 (NEB5) and B. thuringensis NEB17 (NEB17).

B. japonicum was cultured in flasks on a shaker at 200 rev min^{-1,} 50-75 ml in 250 ml flasks or 100-120 ml in 500 ml flasks, at 28° C. in yeast extract mannitol (YEM) culture medium (Vincent, 1970). The initial culture time in flasks inoculated from cold slants was approximately 7 days. The subculture time was not less than 72 h. The cell density in the culture was determined by spectrophotometry at 620 nm, taking A₆₂₀ reading 0.08 as approximately 10⁸ cells ml⁻¹ (Bhuvaneswari et al., 1980). The Bacillus strains were cultured on a shaker at 200 rev min⁻¹ in flasks, 80-100 ml per 250 ml flask or 150-180 ml per 500 ml flask, at 28° C. The culture medium for Bacillus culture was King's Medium B (Atlas, 1995). The initial culture time in flasks inoculated with cold slants was approximately 72 h. The subculture time was 30 h. After the bacterial subcultures were harvested and the cell concentration was determined at 420 nm (Dashti et al., 1997).

The bacterial cultures were diluted with distilled water. The inoculants were prepared by mixing B. japonicum and one of the three tested Bacillus strains. The cell density in the inoculants was 108 cells ml-¹ for both B. japonicum and the co-inoculated Bacillus strain. Under greenhouse conditions the inoculants were applied immediately after preparation, while for the fieldwork there was a delay of not more than 24 h.

Green House Experiment

In the greenhouse experiments the only B. japonicum strain used was 532C (Hume and Shelp, 1990). The greenhouse conditions were: air temperature of 25+2° C., additional illumination of 300 μmol m⁻² s⁻¹ supplied by high pressure sodium lamps (P. L. Light System Canada) for a photoperiod of 16:8 h (day: night). Soybean seeds were surface sterilized in sodium hypochloride (2% solution containing 4 ml Tween20 l⁻¹). The seeds were then rinsed several times with distilled water. The seeds were first planted in trays containing Vermiculite and germinated in the greenhouse. Three or four day old seedlings at the VE stage (Fehr et al., 1971) were transplanted into pots filled with Vermiculite (one seedling per pot) or growth pouches (15×16 cm, Mega International, Minneapolis, Minn., one seedling per pouch). When pouch culture was adopted, the RZT was controlled by water bath systems at 25, 20 and 15° C. respectively (Zhang et al., 1996). Six days after transplanting the seedlings, they were inoculated with the 532C-NEB mixtures at the rate of 1 ml plants⁻¹. Control plants were inoculated with 532C alone or a mixture of 532C and King's Medium B (without bacteria).

The pot experiment was arranged as a completely randomized design (Mead et al.,1993). The pouch experiment was organized following a completely randomized split plot design (Mead et al., 1993). The main plots were RZTs. NEB coinoculation treatments formed the sub-plots. During the growth process, the plants were watered with modified N-free Hoagland's solution (Hoagland and Arnon, 1950), in which Ca(NO₃)₂ and KNO₃ were replaced with 1 mM CaCL₂, 1 mM K₂HPO₄ and 1 mM KH₂PO4, to provide a nitrogen-free solution. The plants were harvested at 55 days after inoculation (DAI). After harvesting, data on nodule number, nodule weight, shoot weight and root weight were collected. All the samples were weighed after not less than 48 h of drying at 70-80° C. The plant weight in greenhouse experiment was calculated as shoot weight plus root weight.

Field Experiment

The field experiment was structured following a completely randomized factorial (3×4) design (Mead et al., 1993) with 3 replications. The tested factors were bradyrhizobial inoculant levels (no inoculant control in which the indigenous B. japonicum community was relied upon for nodulation, B. japonicum 532C and B. japonicum USDA110), and four NEB inoculant levels (no NEB as a control, NEB4, NEB5 and NEB17). The experiment was conducted at the Emile A. Lods Research Centre of McGill University, on a clay-loam type soil where the previous crop was corn, and in 2000 on a sandy-loam type soil where the previous crop was barley. The soybean cultivar was OAC Bayfield. Each plot was 5×1.6 m with 0.2 m between adjacent plots. The plant population was 400 plants plot⁻¹ (500,000 plants ha⁻) with 10 cm between plants within the row and 20 cm between rows. The sowing date was May 20 in 1999 and May 17 in 2000. The soybean seed was sown mechanically. The seeds in the furrows were not covered until the inoculants were added. The inoculants were sprayed into the open furrows by hand, using 60 ml sterilized plastic syringes. The inoculation dose for all inoculants was 1 ml seed⁻.

The plants were harvested three times during whole growing season, at V3, R3 and harvest maturity (R8) stages (Fehr et al. 1971). At the first and second harvest, 5 plants were randomly taken from each plot. After washing the roots with tap water, data on nodule number, nodule weight, shoot weight and root weight were collected in the same way as for greenhouse samples. At the final harvest, plants in the central 1 m of each of the two center rows (an area of 0.4 m²) of each plot were collected by hand. Plant number was determined, and branch number and pod number were counted for each plant. After the roots were detached, the shoots were oven dried at 70-80° C. for not less than 48 h. The shoot weight, including the seeds, was taken as the total weight, i.e. the biological yield or total aboveground biomass. The shoots were mechanically threshed to remove the seeds. The seed weight and the 100-seed weight were also determined. The seed weight was taken as the economic yield. Seed yield is given at 0% moisture. Stem weight was calculated as the difference between the shoot weight and seed weight. The harvest index was expressed as the ratio of the economic yield (the seed weight) to the biological yield (the total weight or total aboveground biomass). The total number of seeds and the seed number per pod were calculated using the variables seed weight, 100-seed weight and pod number. The nitrogen concentrations (%) of the stem and the seed were determined separately using an Element Analyzer (NC2500 Elementary Analyzer, ThermoQuest Italic S.P.A., Italy). The nitrogen yield in stem or seed was calculated by stem or seed weight times their respective nitrogen concentration. The total nitrogen yield was defined as a sum of stem and seed nitrogen yields.

Data Analysis

All the data collected in greenhouse or field experiments were analyzed with the SAS system (Littell et al., 1991). When analysis of variance indicated differences among means, comparisons among the treatment means were conducted with an ANOVA protected least significance difference (LSD) test (Steel and Torrie, 1980). In general, differences were considered significant when detected at P≦0.05. However, in some cases differences at 0.05<P<0.1 are discussed in the text. When this happens the P value is provided.

Results

Greenhouse Experiment

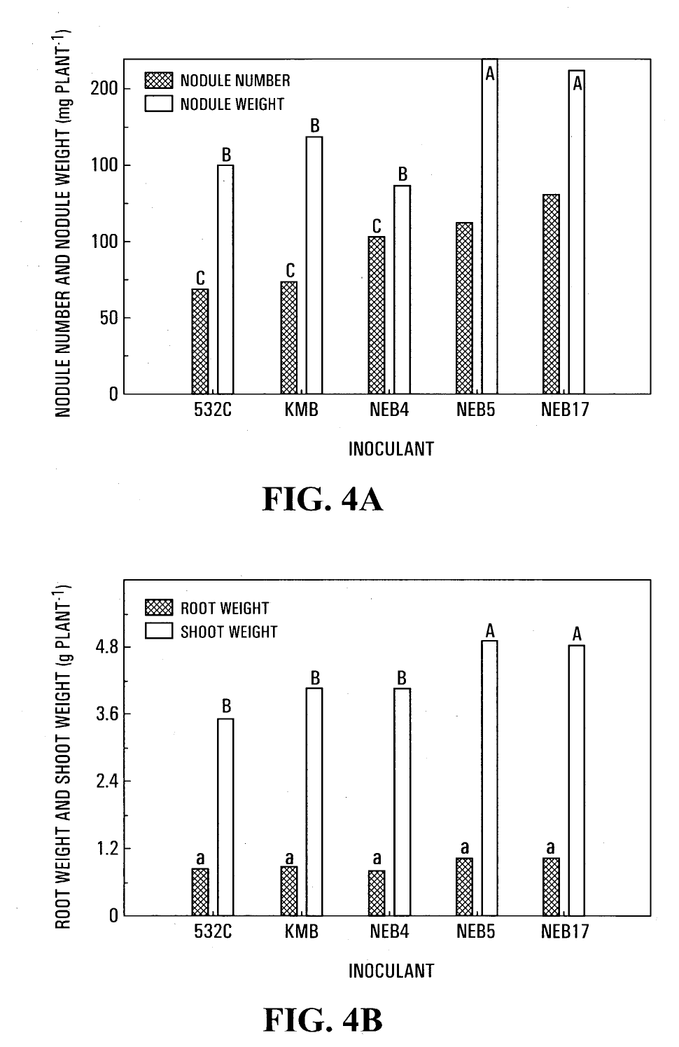

The general patterns of responses to the treatments were the same in growth pouch and pot culture systems. In pot experiment, coinoculation of NEB5 and NEB17 increased nodule number, nodule weight and shoot weight, whereas coinoculation of NEB4 failed to increase shoot weight (FIG. 4). In pouch experiment, compared with 25° C., the optimal RZT for soybean growth and nodulation, 15° C. RZT greatly inhibited the plant nodulation and growth, while 20° C. RZT had little inhibitory effect (Table 1). Coinoculation of the three Bacillus NEB strains generally promoted soybean plant growth and nodulation under either optimal or suboptimal RZT conditions (Table 1). Coinoculation of NEB 17 resulted in constant plant growth promotion, regardless of RZT (Table 1), whereas responses to coinoculation with NEB4 and NEB5 were less consistent. Inclusion of NEB17 in the inoculant resulted in increases in nodule number, nodule weight, shoot weight and root weight. NEB5 performed almost as well as NEB17. NEB4 stimulated nodule number and shoot weight at 15° C. RZT, and root weight and shoot weight at 20° C. RZT, but had no effect on the four measured variables at 25° C. RZT (Table 1). In both pouch and pot experiments, the two controls were not different from each other.

Field Experiments (Bacillus Strains)

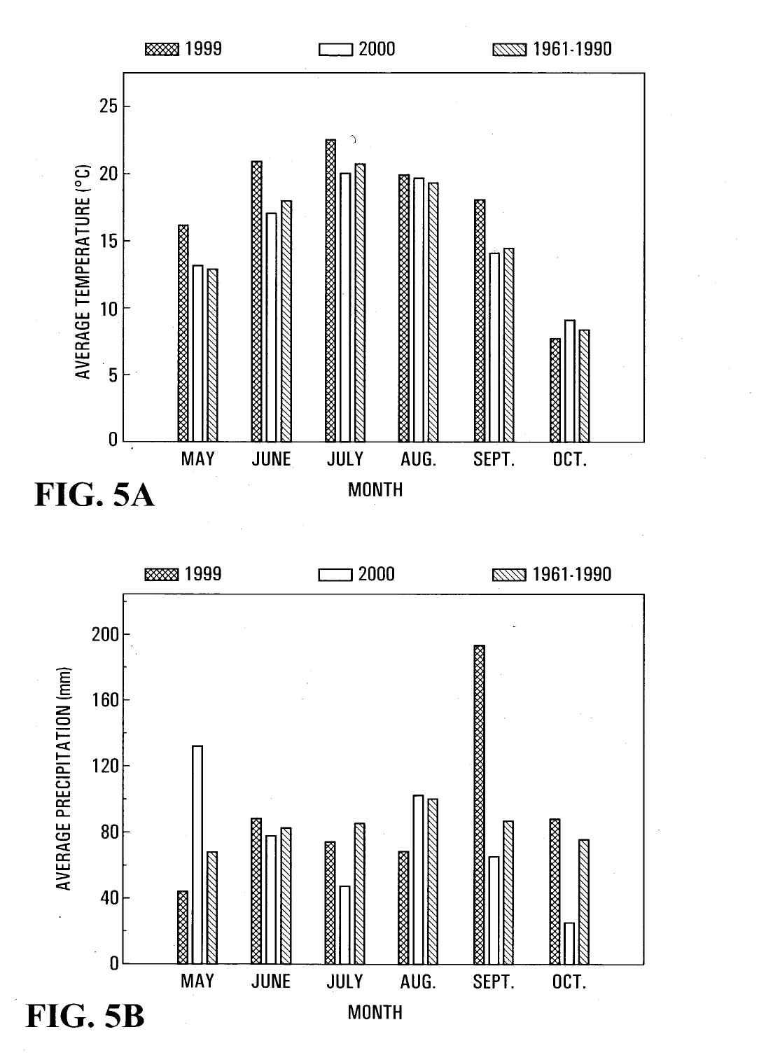

Under field conditions, there were no interactions between B. japonicum or among Bacillus NEB strains. This occurred in spite of the different growth conditions (soil types and weather, FIG. 5), in Year 1 and Year 2, which resulted in different levels of overall plant growth. Comparatively speaking, the general growth conditions in Year 2 were better than in Year 1. In Year 1 the average total biomass production was 10.08 t ha⁻¹ and seed production was 5.33 t ha⁻¹ compared to 13.95 t ha⁻¹ and 7.83 t ha⁻¹, respectively, in Year 2. Similar differences also existed when the respective within growth season harvests (at V3 and R3 stages) were compared across years. However, the relative performances of the treatments were similar in both years (Table 2).

At both V3 and R3 stages, nodule number, nodule weight and plant weight were increased by coinoculation of all the three NEB strains (Table 2). None of the three selected NEB strains had any negative effects on soybean plant growth and nodulation. At the V3 stage, the nodule number was increased by 34.7% (NEB17, Year 2) to 185% (NEB4, Year 1); the nodule weight was increased by 21.5% (NEB4, Year 2) to 36.8% (NEB17, Year 1); and the plant weight was increased by 6.4% (NEB5, Year 1) to 64.1% (NEB17, Year 2). At the R3 stage, the nodule number was increased in 46.1% (NEB17, Year 2) to 66.3% (NEB17, Year 1); the nodule weight was increased by 27.1% (NEB4, Year 1) to 69.6% (NEB5, Year 2); and the plant weight was increased by 6.5% (NEB5, Year 1) to 52.7% (NEB5, Year 2). These data show that the three co-inoculated NEB strains were all reasonably effective in promoting plant growth up to the R3 stage.

As at the V3 and R3 stages, all the measured variables at the final harvest were larger in Year 2 than in Year 1 (Table 3). However, in Year 1, coinoculation of each NEB strain increased total weight (P=0.08) by 13.2 to 16.6%, and seed weight by 14.9 to 16.5%. In Year 2, only the coinoculation of NEB17 increased total weight (27.3%) and seed weight (P=0.07, 22.9%). In Year 2, the total seed number was increased in parallel with seed weight due to coinoculation of NEB17.

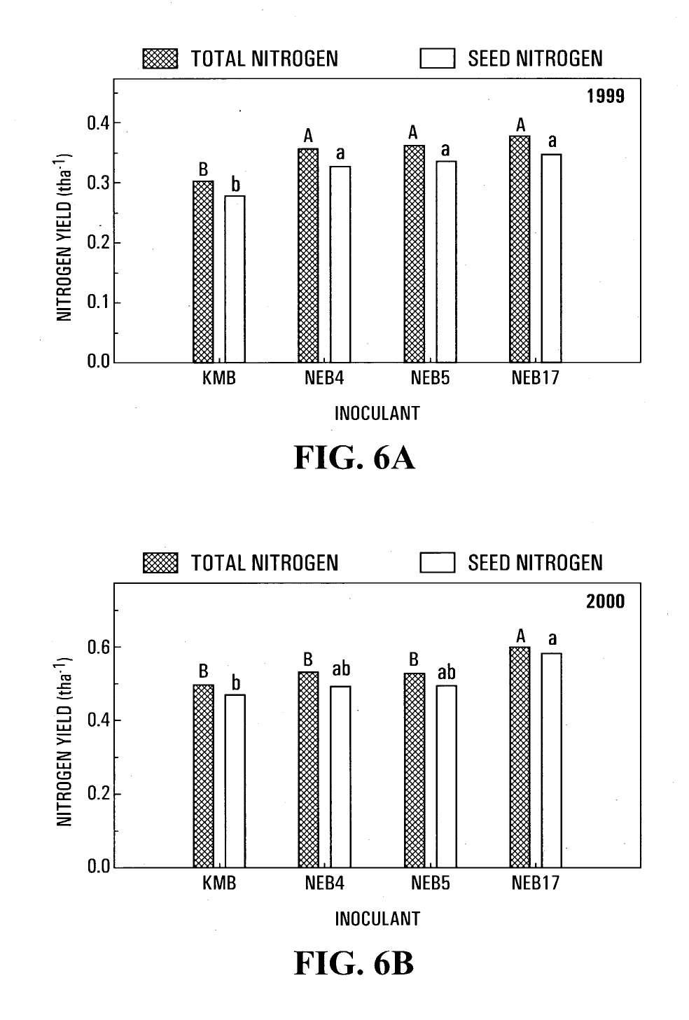

In both years the nitrogen concentration (%) of either stems or seeds were not different among the treatments. In Year 1, the stem nitrogen concentration was between 0.58-0.62% and seed nitrogen concentration between 5.77-6.19%. In Year 2, the stem nitrogen concentration was between 0.52-0.62% and seed nitrogen concentration between 6.36-6.62%. The total nitrogen yield and the seed nitrogen yield (FIG. 6) paralleled the biological and the economic yields (Table 3). In Year 1, coinoculation of the three NEB strains resulted in increases in total nitrogen and seed nitrogen yield, relative to the control. Among the PGPB treatments, NEB17 caused the greatest responses, increasing the total nitrogen yield by 24.8% and the seed nitrogen yield by 22.3%. In Year 2, only coinoculation of NEB17 increased the total nitrogen yield by 25.8% and the seed nitrogen content by 23.4% (P=0.07) over the control.

Field Experiments (B. Japonicum Strains)

At the final harvests, there were few differences among the three B. japonicum levels (no-inoculant,

B. japonicum 532C and B. japonicum USDA110) in Year 2 (Table 4). In Year 1, both the inoculated bradyrhizobial strains increased biological and economic yields relative to the control (no-inoculant) (P=0.10).

TABLE 1
Pouch experiment results for the three Bacillus NEB strains at three root
zone temperatures (RZTs) under greenhouse conditions

| | Nodule number | Nodule weight | Root weight | Shoot weight |
|---|---|---|---|---|
| Treatment | (per plant) | (g plant⁻¹) | (g plant⁻¹) | (g plant⁻¹) |
| 15° C. RZT | | | | |
| 532C Control | 14.5 c | 0.019 b | 0.224 b | 0.601 b |
| 532C + King's Medium | 16.4 c | 0.019 b | 0.230 b | 0.601 b |
| B | | | | |
| 532C + NEB4 | 24.3 b | 0.024 ab | 0.274 ab | 0.741 a |
| 532C + NEB5 | 25.0 b | 0.027 a | 0.296 a | 0.752 a |
| 532C + NEB17 | 30.9 a | 0.029 a | 0.300 a | 0.746 a |
| 20° C. RZT | | | | |
| 532C Control | 31.8 b | 0.032 d | 0.244 b | 0.820 b |
| 532C + King's Medium | 32.6 b | 0.036 cd | 0.249 b | 0.802 b |
| B | | | | |
| 532C + NEB4 | 34.0 b | 0.040 bc | 0.337 a | 1.088 a |
| 532C + NEB5 | 45.2 a | 0.046 ab | 0.353 a | 1.131 a |
| 532C + NEB17 | 45.2 a | 0.048 a | 0.345 a | 1.115 a |
| 25° C. RZT | | | | |
| 532C Control | 32.6 b | 0.034 c | 0.250 b | 0.828 c |
| 532C + King's Medium | 34.3 b | 0.036 c | 0.268 ab | 0.841 c |
| B | | | | |
| 532C + NEB4 | 37.3 ab | 0.041 bc | 0.319 ab | 0.944 bc |
| 532C + NEB5 | 43.0 a | 0.048 b | 0.331 a | 1.091 ab |
| 532C + NEB17 | 44.0 a | 0.051 a | 0.327 a | 1.186 a |

Treatment: 532C, Bradyrhizobium japonicum 532C; NEB4 and NEB5, Bacillus subtilis strains; NEB17, Bacillus thuringiensis strain. The plants were harvested at 55 days after inoculation. Within the same column and root zone temperature, means followed by the same letter are not different (p = 0.05) by an ANOVA protected LSD test. n = 6.

TABLE 2
Main effects of co-inoculation of NEB strains with Bradyrhizobium japonicum
strains on nodulation variables at V3 and R3 stages of field grown soybean
plants in 1999 and 2000

| | Year 1 experiment | | | Year 2 experiment | | |
|---|---|---|---|---|---|---|
| | Nodule | Nodule | Plant | Nodule | Nodule | Plant |
| | number | weight | weight | number | weight | weight |
| Inoculant | (per plan) | (g plant⁻¹) | (g plant⁻¹) | (per plant) | (g plant⁻¹) | (g plant⁻¹) |
| V3 stage harvest | | | | | | |
| Control | 8.5 b | 0.068 b | 3.75 a | 35.7 b | 0.214 b | 3.84 b |
| NEB4 | 24.3 a | 0.086 a | 4.29 a | 51.1 ab | 0.293 a | 5.77 a |
| NEB5 | 21.9 a | 0.091 a | 3.99 a | 55.3 a | 0.300 a | 6.17 a |
| NEB17 | 23.9 a | 0.093 a | 4.17 a | 48.1 ab | 0.339 a | 6.30 a |
| R3 stage harvest | | | | | | |
| Control | 37.2 c | 0.380 c | 20.96 c | 49.9 b | 0.519 b | 22.46 b |
| NEB4 | 44.9 b | 0.483 b | 23.70 ab | 82.4 a | 0.814 a | 32.81 a |
| NEB5 | 49.7 b | 0.453 ab | 22.33 bc | 89.6 a | 0.880 a | 34.29 a |
| NEB17 | 62.2 a | 0.510 a | 24.23 a | 72.9 ab | 0.748 a | 31.71 ab |

Inoculant: Control, NEB4, NEB5 and NEB17, same as those in FIG. 3. Within the same column and harvest stage, means followed by the same letter are not different (P = 0.05) by an ANOVA protected LSD test. n = 9.

TABLE 3
Main effects of coinoculation of NEB strains with Bradyrhizobium japonicum
strains on yield and yield components of field grown soybean plants at
harvest maturity in Year 1 and Year 2

| | Total | Pod number | Total seed | | 100-seed | |
|---|---|---|---|---|---|---|
| | weight | (10⁶ | (10⁶ | Seed weight | weight | Harvest |
| Inoculant | (t/ha) | pods/ha) | seeds/ha) | (t/ha) | (g) | index |
| Year 1 | | | | | | |
| experiment | | | | | | |
| Control | 9.12 b | 11.10 | 26.17 | 4.82 b | 18.46 | 0.529 |
| NEB4 | 10.63 a | 13.08 | 29.21 | 5.54 a | 19.05 | 0.521 |
| NEB5 | 10.76 a | 12.10 | 28.75 | 5.61 a | 19.55 | 0.534 |
| NEB17 | 10.34 a | 12.86 | 29.76 | 5.61 a | 18.93 | 0.545 |
| Year 2 | | | | | | |
| experiment | | | | | | |
| Control | 12.37 b | 14.46 | 34.24 b | 7.22 b | 21.07 | 0.559 |
| NEP4 | 13.37 ab | 15.62 | 36.30 b | 7.77 b | 21.40 | 0.566 |
| NEB5 | 13.28 b | 15.42 | 33.57 b | 7.44 b | 31.34 | 0.560 |
| NEB17 | 15.74 a | 17.11 | 41.26 a | 8.87 a | 21.53 | 0.584 |

Inoculant: Control, NEB4, NEB5 and NEB17, same as in FIG. 7.3. Within the same column and experiment, means followed by the same letter are not different ( at P = 0.05 level in Year 1 experiment and at P = 0.10 level in Year 2 experiment by an ANOVA protected LSD test. Where columns are not followed by letters the means were not different (P = 0.10). n = 9.

TABLE 4
Main effects of Bradyrhizobium japonicum inoculants on
variables related to dry matter and nitrogen yield of
soybean plants grown under field conditions
in Year 1 and Year 2.

| | Total | Seed | Total | Seed |
|---|---|---|---|---|
| | weight | weight | Nitrogen | Nitrogen |
| | (t ha⁻¹) | (t ha⁻¹) | (t ha⁻¹) | (t ha⁻¹) |
| Year 1 experiment | | | | |
| No-inoculant control | 8.36 b | 4.44 b | 0.278 | 0.252 |
| B. japonicum USDA110 | 10.06 a | 5.37 a | 0.353 | 0.324 |
| B. japonicum 532C | 10.42 a | 5.51 a | 0.353 | 0.325 |
| Year 2 experiment | | | | |
| No-inoculant control | 13.30 | 7.54 | 0.512 | 0.477 |
| B. japonicum USDA110 | 13.97 | 7.79 | 0.544 | 0.509 |
| B. japonicum 532C | 14.18 | 8.15 | 0.576 | 0.541 |

Within the same column and year, means followed by the same letter are not different at p = 0.10 level by an ANOVA protected
LSD test. When there were no differences among the means there are no letters following the means of that year. n = 12.

### Deposits of Biological Materials

The following deposits of biological materials were made pursuant to the terms of the Budapest Treaty on the International Recognition of the Deposit of Microorganisms for the Purposes of Patent Procedure at the International Depositary Authority of Canada, Bureau of Microbiology, Health Canada, 1015 Arlington Street, Winnipeg, Manitoba Canada, R3E 3R2:

Bacillus subtilis strain NEB4 was deposited on Mar. ______, 2003 under Accession No. ______.

Bacillus subtilis strain NEB5 was deposited on Mar. ______, 2003 under Accession No. ______.

Bacillus thuringiensis strain NEB17 was deposited on Mar. ______, 2003 under Accession No. ______.

### REFERENCES

Altschul, S. F., Madden, T. I., Scaffer, A. A., Zhang, J., Zhang, Z., Miller, W., and Lipman, D. J. 1997. Gapped BLAST and PSI-BLAST: a new generation of protein database search program. Nucleic Acids Res. 25: 3389-3402.

Atlas, R. M. 1995. Handbook of media for environmental microbiology. CRC Press. Boca Raton, Fla. USA. pp. 32-33, 237-238.

Bhuvaneswari T V, Googman R N, Bauer W D. 1980. Early events in the infection of soybean [Glycine max (L.) Merr.] by Rhizobium japonicum. I. Location of infectible root cells. Plant Physiology 66: 1027-1031.

Bremner, J. M. 1965. Total nitrogen. In Methods of soil analysis (2). Edited by C. A. Black. American Society of Agronomy, Madison, Wis. pp. 1149-1178.

Dashti, N., Zhang, F., Hynes, R., and Smith, D. L. 1997. Application of plant growth-promoting rhizobacteria to soybean [Glycine max (L.) Merrill] increases protein and dry matter yield under short-season conditions. Plant Soil, 188:33-41.

Fehr, W. R., Caviness, C. E, Burmood, D. T., and Pennington, J. S. 1971. Stages of development descriptions for soybeans, [Glycine max (L.) Merrill]. Crop Sci. 11: 929-931.

Goto, K., Omura, T., Hara, Y., and Sadaie, Y. 2000. Application of the partial 16S rDNA sequence as an index for rapid identification of species in the genus Bacillus. J. Gen. Appl. Microbiol. 46: 1-8.

Hoagland, B., and Arnon, D. I. 1950. The water culture method for growing plants without soil. Calif. Agri. Exp. Sta. Cir. 347: 23-32.

Hume, D. L., and Shelp, B. J. 1990. Superior performance of the Hup Bradyrhizobium japonicum strain 532C in Ontario soybean field trials. Can. J. Plant Sci. 70: 661-666.

Kobayashi, D. Y., and Palumbo, J. D. 2000. Bacterial endophytes and their effects on plants and uses in agriculture. In Microbial endophytes. Edited by C. W. Bacon and J. F. White. Marcel Dekker, Inc., New York. pp. 199-233.

Littell R C, Freund R J, Spector P C. 1991. SAS System for Linear Models. 3rd edn. Cary, N.C.: USA.SAS Institute Inc.

Mead, R., Curnow, R. N., and Hasted, A. M. 1993. Statistical methods in agriculture and experimental biology. 2nd ed. Chapman & Hall. London.

Misaghi, I. J., and Donndelinger, C. R. 1990. Endophytic bacteria in symptom-free cotton plants. Phytopathology, 80: 808-811.

Ritchie, D. A., Edwards, C., McDonald, I. R., and Murrell, J. C. 1997. Detection of methanogens and methanotrophs in natural environments. Glob. Change. Biol. 3: 339-350.

Saitou, N., and Nei, M. 1987. The neighbor-joining method: a new method for reconstructing phylogenetic trees. Mol. Biol. Evol. 4: 406-425.

Sambrook, J., Fritsch, E. F., and Maniatis, T. 1989. Molecular cloning: a laboratory manual, 2nd ed. Cold Spring Harbor Press, Cold Spring Harbor, N.Y.

Steel, R. G. D., and Torrie, J. H. 1980. Principles and procedures of statistics: A biometric approach. McGraw-Hill, New York.

Tamura, K., and Nei M. 1993. Estimation of the number of nucleotide substitutions in the control region of mitochondrial DNA in humans and chimpanzees. Mol. Biol. Evol. 10: 512-526.

Thompson, J. D., Higgins, D. G., and Gibson, T. J. 1994. CLUSTAL W: Improving the sensitivity of progressive multiple sequence alignment through sequence weighting, positions-specific gap penalties and weight matrix choice. Nucleic Acids Res. 22: 4673-4680.

Vincent, J. M. 1970. A manual for the practical study of root nodule bacteria. Blackwell Scientific Publications, Oxford. pp. 169-170.

Zhang, F., Dashti, N., Hynes, H., and Smith, D. L. 1996. Plant growth promoting rhizobacteria and soybean [Glycine max (L.) Merr.] nodulation and nitrogen fixation at suboptimal root zone temperatures. Ann. Bot. 77: 453-459.

1. A method for increasing plant growth, comprising:
inoculating a plant with plant growth promoting bacteria selected from the group consisting of plant growth promoting bacteria of the species Bacillus subtilis and plant growth promoting bacteria of the species Bacillus thuringiensis or a combination thereof.

2. The method according to claim 1, wherein said plant growth promoting bacteria have a partial 16S ribosomal RNA gene sequence possessing at least 60% sequence identity to SEQ ID NO:1, SEQ ID NO:2 or SEQ ID NO:3.

3. The method according to claim 1, wherein plant growth promoting bacteria are selected from the group consisting of B. subtilis having the identifying characteristics of B. subtilis strain NEB4, B. subtilis having the identifying characteristics of B. subtilis strain NEB5, and B. thuringiensis having the identifying characteristics of B. thuringiensis strain NEB17.

4. The method according to claim 1, wherein the plant growth promoting bacteria are selected from the group consisting of B. subtilis strain NEB4, B. subtilis strain NEB5, and B. thuringiensis strain NEB17.

5. The method according to claim 1, further comprising inoculating said plant with nitrogen-fixing rhizobacteria.

6. The method according to claim 5, wherein said nitrogen-fixing rhizobacteria comprise bacteria of the genus Bradyrhizobium.

7. The method according to claim 5, wherein said nitrogen-fixing rhizobacteria comprise bacteria of the species Bradyrhizobium japonicum.

8. The method according to claim 1, wherein said inoculating is effected by an inoculation method selected from the group consisting of:
(a) inoculating soil with said plant growth promoting bacteria prior to or contemporaneously with sowing plant seeds in said soil;
(b) applying said plant growth promoting bacteria to plant seeds prior to or at the time of sowing said seeds; and,
(c) applying said plant growth promoting bacteria to soil after plant seeds have been sown in said soil, or to growing plants or to the roots, stems, leaves or other parts thereof.

9. The method according to claim 1, wherein said plant is a legume.

10. The method according to claim 9, wherein said legume is a soybean.

11. An inoculant for increasing plant growth, comprising plant growth promoting bacteria selected from the group consisting of plant growth promoting bacteria of the species Bacillus subtilis and plant growth promoting bacteria of the species Bacillus thuringiensis, or a combination thereof.

12. The inoculant according to claim 11, wherein said plant growth promoting bacteria have a partial 16S ribosomal RNA gene sequence possessing at least 60% sequence identity to SEQ ID NO:1, SEQ ID NO:2 or SEQ ID NO:3.

13. The inoculant according to claim 12, wherein said plant growth promoting bacteria are selected from the group consisting of B. subtilis having the identifying characteristics of B. subtilis strain NEB4, B. subtilis having the identifying characteristics of B. subtilis strain NEB5, and B. thuringiensis having the identifying characteristics of B. thuringiensis strain NEB27.

14. The inoculant according to claim 12, wherein said plant growth promoting bacteria are selected from the group consisting of B. subtilis strain NEB4, B. subtilis strain NEB5, and B. thuringiensis strain NEB27.

15. The inoculant according to claim 11, further comprising nitrogen-fixing rhizobacteria.

16. The inoculant according to claim 15, wherein said nitrogen-fixing rhizobacteria comprise bacteria of the genus Bradyrhizobium.

17. The inoculant according to claim 15, wherein said nitrogen-fixing rhizobacteria comprise bacteria of the species Bradyrhizobium japonicum.

18. A kit for increasing plant growth, comprising: an inoculant according to claim 11, and instructions for use of said inoculant for promoting plant growth.

19. A biologically pure culture of plant growth promoting bacteria selected from the group consisting of:
(a) a biologically pure culture of Bacillus subtilis having the identifying characteristics of B. subtilis strain NEB4;
(b) a biologically pure culture of Bacillus subtilis, having the identifying characteristics of B. subtilis strain NEB5; and
(c) a biologically pure culture of Bacillus thuringiensis having the identifying characteristics of B. thuringiensis strain NEB17.

20. The biologically pure culture of plant growth promoting bacteria according to claim 19, selected from the group consisting of:
(a) a biologically pure culture of B. subtilis strain NEB4;
(b) a biologically pure culture of B. subtilis strain NEB5; and
(c) a biologically pure culture of B. thuringiensis strain NEB17.
